(19) United States
(12) Patent Application Publication
(10) Pub. No.: US 2011/0243959 A1
(43) Pub. Date: Oct. 6, 2011

(54) MODULATING NEURONAL PLASTICITY AND TREATMENT OF NEURONAL LOSS BY MODULATIONG WNT7A OR WNT7B MODULATION

(76) Inventor: Pico Caroni, Muttenz (CH)

(21) Appl. No.: 13/126,585

(22) PCT Filed: Nov. 3, 2009
(86) PCT No.: PCT/EP2009/064504

§ 371 Date: Jun. 1, 2011

(30) Foreign Application Priority Data
Nov. 4, 2008 (EP) 08168278.3

(51) Int. Cl. A61K 39/395; A61K 31/7115; A61P 25/28; A61P 25/00

(52) U.S. Cl. 424/158.1; 514/44 A

(57) ABSTRACT

The present invention relates to a method of treating or preventing a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, of modulating neuronal activity and/or neuronal plasticity, of optimizing repair after brain injury, to modulate learning, and/or to modulate cognitive capabilities, in a subject and/or, of preventing loss of neuronal connections and/or to increase neuronal connections in said subject, by modulating Wnt-signaling, which method is characterized by the fact that the subject is in puberty or post-puberty, wherein puberty or post-puberty means that the neuronal circuits of said subject have already undergone developmental synapse elimination and critical periods which take place prior to puberty.

### FIELD OF THE INVENTION

The present invention relates to a method of treating or preventing a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, of modulating neuronal activity and/or neuronal plasticity, of optimizing repair after brain injury, to modulate learning, and/or to modulate cognitive capabilities, in a subject and/or, of preventing loss of neuronal connections and/or to increase neuronal connections in said subject.

### BACKGROUND OF THE INVENTION

The nervous system recruits a rich repertoire of plasticity mechanisms in order to interact successfully with its environment. Among them, synaptic plasticity includes modifications of existing synapses, and the formation and loss of synaptic connections (i.e. structural plasticity). While it is clear that structural plasticity could alter the function of neuronal circuits in profound ways, the precise changes that do take place in healthy adults have remained unclear (Chklovskii et al., 2004; Alvarez and Sabatini, 2007; Gogolla et al., 2007). Time-lapse imaging studies in vivo have revealed the existence of subpopulations of dynamic synapses, which form and disassemble with half-lifes of days or weeks in adult mice and primates. This ongoing structural plasticity involves synaptic rearrangements that would not be detectable without repeated observations of the same sets of synapses, and appear to undergo homeostatic control of synapse numbers (Alvarez and Sabatini, 2007; Gogolla et al., 2007). In contrast to these subtle synaptic rearrangements, quantitative differences in dendritic or axonal structures have been detected in animals that had undergone hormonal status changes, repeated training for new skills, environmental enrichment, and/or chronic stress (Moser et al., 1997; McEwen, 1999; Sandi et al., 2003; Stewart et al., 2005; Fuchs et al., 2006; Galimberti et al., 2006).

Due to its unique anatomical features, the mossy fiber projection by dentate gyrus (DG) granule cells onto hippocampal CA3 has provided an advantageous model system to investigate axonal structural plasticity in the adult. The axons of glutamatergic granule cells (a.k.a. mossy fibers) synapse onto excitatory and inhibitory interneurons in the hilus, and then extend in tight lamellar bundles throughout stratum lucidum in CA3, to establish 8-10 Large Mossy fiber Terminals (LMTs) onto pyramidal neurons (Claiborne et al., 1986; Henze et al., 2000; Rollenhagen et al., 2007). Anatomically, LMTs are terminal bouton structures, which consist of a “parent LMT” associated with the main axon, and can include “satellite LMTs”, which are connected by thin processes to the parent LMT (Galimberti et al., 2006; Gogolla et al., 2007). Mossy fibers further synapse onto inhibitory neurons in CA3 through en-passant boutons and filopodial extensions of the LMTs (Acsady et al., 1998; McBain, 2008). In addition to their low numbers, large sizes and lamellar distributions, the potent synaptic connections by mossy fibers onto pyramidal neurons relate in a dramatic fashion structure to function. This is due to the large number of synapses which individual LMTs can establish with postsynaptic thorny excrescences of individual excitatory pyramidal neurons, and the increasing co-operativity of these synapses at higher spiking frequencies (Gonzales et al., 2001; Henze et al., 2002; Lawrence and McBain, 2003; Mon et al., 2004; Kobayashi and Poo, 2004; Nicoll and Schmitz, 2005; Engel and Jonas, 2005; Rollenhagen et al., 2007; McBain, 2008).

The connectivity between mossy fiber LMTs and CA3 pyramidal neurons exhibits pronounced structural plasticity in the adult. Most dramatically, adult neurogenesis of granule cells leads to the experience-related incorporation of new projections throughout life (van Praag et al., 2000; Ninkovic et al., 2007). In addition, hippocampal learning can produce spatial expansions of the mossy fiber projection, whereas chronic stress produces global reductions in postsynaptic spine densities (Schopke et al., 1991; Pleskacheva et al., 2000; Ramirez-Amaya et al., 2001; Sandi et al., 2003; Stewart et al., 2005). Investigations based on the visualization of individual axons and dendrites in transgenic mice expressing GFP constructs in few neurons have provided more precise accounts of this structural plasticity. Thus, time-lapse imaging studies in organotypic slice cultures have provided evidence for balanced turnover of mossy fiber boutons regulated by AMPA receptor activation (De Paola et al., 2003). In addition, a detailed analysis of mossy fiber connectivities in situ, has provided evidence for macroscopically detectable long-term alterations in the adult: housing mice under enriched environment (EE) conditions produced a marked increase in dendritic spine lengths and “satellite LMTs”, whereas a gradual life-long shift in LMT size distributions produced increasingly asymmetric distributions of LMT sizes as a function of increasing age (Galimberti et al., 2006).

The cellular and molecular mechanisms involved in the large-scale remodeling of LMTs in the adult have remained unclear. The present inventors have hypothesized that synaptic activity might be involved. Wnt proteins are lipid-modified secreted peptides that mediate local cell signaling through several distinct receptor systems (e.g. Mikels and Nusse, 2006). In addition to their well-known roles in early development, Wnt proteins have recently been shown to have important roles in the assembly and plasticity of central synapses (Salinas, 2005; Salinas and Zou, 2008). Thus, Wnt proteins are expressed in adult neurons, and postsynaptically released Wnt7 proteins promote presynaptic assembly and synaptic vesicle accumulation at maturing cerebellar and hippocampal synapses, and facilitate LTP at hippocampal synapses (Hall et al., 2000; Packard et al., 2002; Ahmad-Annuar et al., 2006; Chen et al., 2006; Abe and Takeichi, 2007; Cerpa et al., 2008; Ataman et al., 2008). A hallmark of Wnt signaling pathways is their high degree of versatility, which is mediated through the cell- and context-specific expression, targeting and activation of specific ligands, receptors and signal tranduction components (Mikels and Nusse, 2006).

In order to confirm their hypothesis and hence provide methods and molecules suitable to influence the plasticity of neuronela connections in adults, the present inventors investigated the synaptic and molecular mechanisms underlying global remodeling of LMTs in response to experience and age in vivo.

### SUMMARY

The present inventors demonstrate that, surprisingly, EE specifically and reversibly produces a robust increase in stratum lucidum synapse numbers, and in the densities of synapses per LMT. In parallel, the EE conditions produce a marked increase in CA3 pyramidal neuron Wnt7a/b protein levels, a member of the Wnt-signaling pathway. Local inhibition of Wnt signaling in CA3 suppresses the effects of EE on synapse numbers, and further reduces synapse numbers in mice housed under control conditions; conversely, the local application of a Wnt-signaling agonist is sufficient to mimick the effects of EE, both in vivo and in slice cultures. Taken together, these surprising results show that Wnt signaling mediates global regulation of synapse numbers in response to experience and age, not only in young animals, i.e. before and during the normal developmental synapse elimination and critical periods, but also in adult hippocampal stratum lucidum. These sustained global alterations in synapse numbers and connectivities hence represent a novel class of structural plasticity mechanisms, which allow to modify network properties in response to experience.

The present invention hence provides a method of preventing and/or treating a disease characterized by a loss or imbalance of neuronal connections in a subject by administering to said subject a therapeutically effective amount of a modulator of Wnt-signaling, wherein (i) said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, and wherein (ii) said subject is in puberty or post-puberty, wherein puberty or post-puberty means that the neuronal circuits of said subject have already undergone developmental synapse elimination and critical periods which take place prior to puberty.

Another embodiment of the invention encompasses a method of modulating neuronal activity and/or neuronal plasticity, optimizing repair after brain injury, modulating learning, and/or modulating cognitive capabilities in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty.

A further embodiment of the present invention is a method of preventing loss of neuronal connections and/or increasing neuronal connections in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty.

In the methods of the invention, Wnt-signaling can be modulated by an agonist of Wnt-signaling, for instance a small molecule, and antibody or a siRNA.

In some embodiments of the invention, the subject is a mammal, for example a human subject. Moreover, in some embodiments, the modulation of the Wnt-signaling can occur in the hippocampus of the subject.

In a particular embodiment of the invention, the expression and/or biological activity of Wnt7a and/or Wnt7b is modulated, for instance increased.

In a further aspect, the present invention encompasses a siRNA decreasing or silencing the expression of an antagonistic component of Wnt-signaling, thus for example increasing the level of Wnt7a and/or Wnt7b, for use as a medicament in a subject who is in puberty or post-puberty, wherein puberty or post-puberty means that the neuronal circuits of said subject have already undergone developmental synapse elimination and critical periods which take place prior to puberty, to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject.

Moreover, the present invention also encompasses an antibody specifically binding to a modulator of Wnt-signaling, for instance an antagonist of Wnt-signalling, for use as a medicament in a subject who is in puberty or post-puberty, wherein puberty or post-puberty means that the neuronal circuits of said subject have already undergone developmental synapse elimination and critical periods which take place prior to puberty to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject. For instance, the antibody of the invention can inhibit the binding of said Wnt-signaling component to its ligand. In some embodiment, the antibody acts as an agonist of Wnt-signalling by acting in an agonistic way on its specific target.

The present invention also encompasses a drug delivery device for implantation into the nervous system of a subject who is in puberty or post-puberty, wherein puberty or post-puberty means that the neuronal circuits of said subject have already undergone developmental synapse elimination and critical periods which take place prior to puberty, the drug delivery device comprising: a biocompatible polymer; and a modulator of Wnt-signaling, for instance an agonist, wherein said modulator of Wnt-signaling is released from the polymer into the nervous system of the subject in an amount effective to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1: EE conditions produce an increase in LMT complexities and LMT numbers.

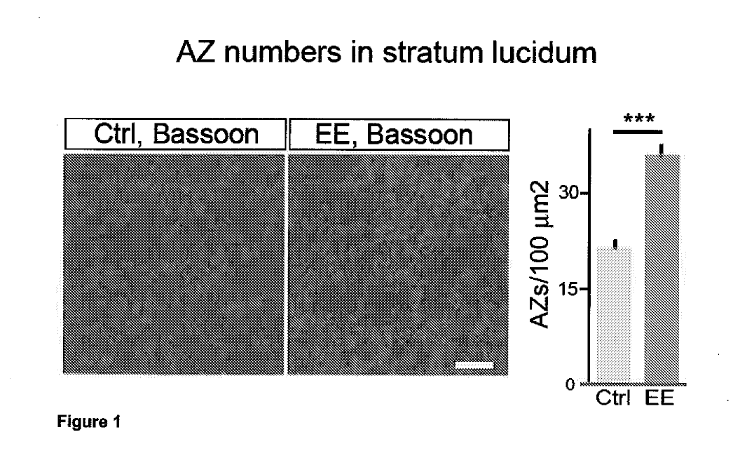

FIG. 2: EE conditions produce a marked increase in stratum lucidum AZ (active zone) numbers.

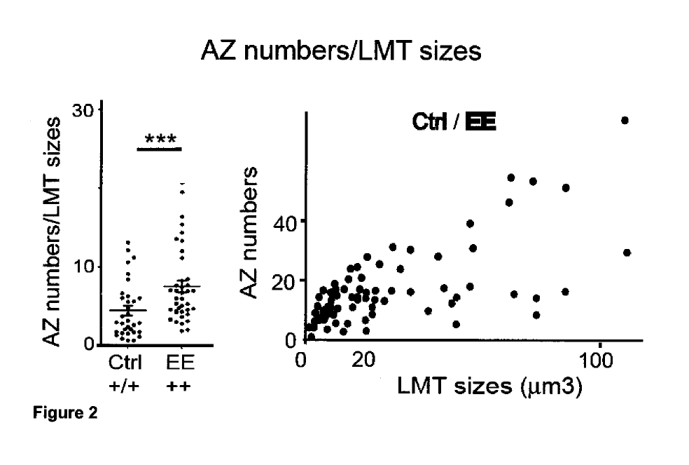

FIG. 3: The increase in AZ numbers does not just reflect the increase in LMT numbers, but involves a specific increase in the numbers of AZs per individual LMT upon EE conditions. Furthermore, the numbers of AZs per LMT are not simply proportional to LMT sizes. Thus, a majority of larger LMTs exhibit substantially lower AZ densities than smaller LMTs under control conditions. This is reflected in the fact that for the subpopulation of LMTs with relatively larger sizes (30-40% of total LMTs), LMT sizes seem to vary independently of AZ numbers. Satellite LMTs did not differ significantly from parent LMTs in their AZ/volume relationship (not shown). Significantly, while the EE conditions produce higher numbers of AZs per volume for most LMTs, this effect of the EE conditions is most pronounced for LMTs of larger sizes.

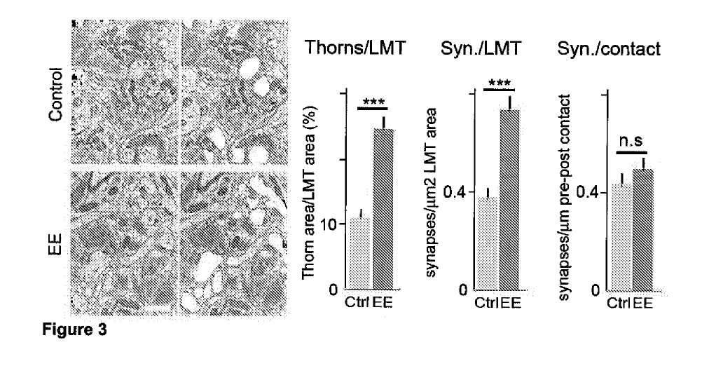

FIG. 4: Housing mice under EE conditions lead to a robust and generalized increase in Wnt7a/b immunoreactivity in CA3.

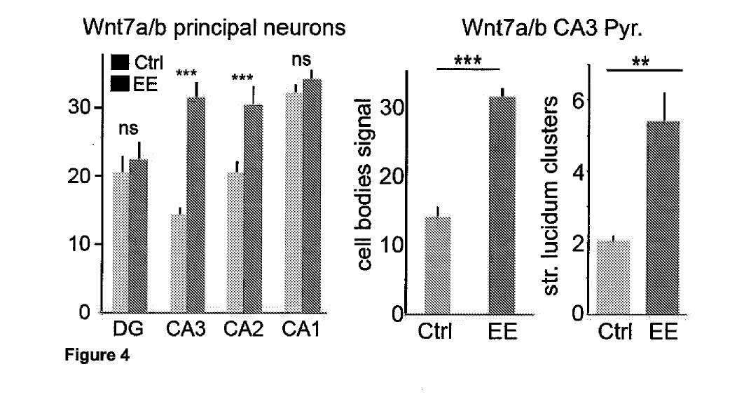

FIG. 5: The presence of secreted frizzled-related protein 1 (sFRP-1) in CA3 (but not in the dentate gyrus; not shown) effectively suppresses the effect of the enriched housing conditions on satellite and AZ numbers. sFRP-1 or vehicle were applied during the entire EE procedure through a canule, targeting their release to a circumscribed region of hippocampal CA3.

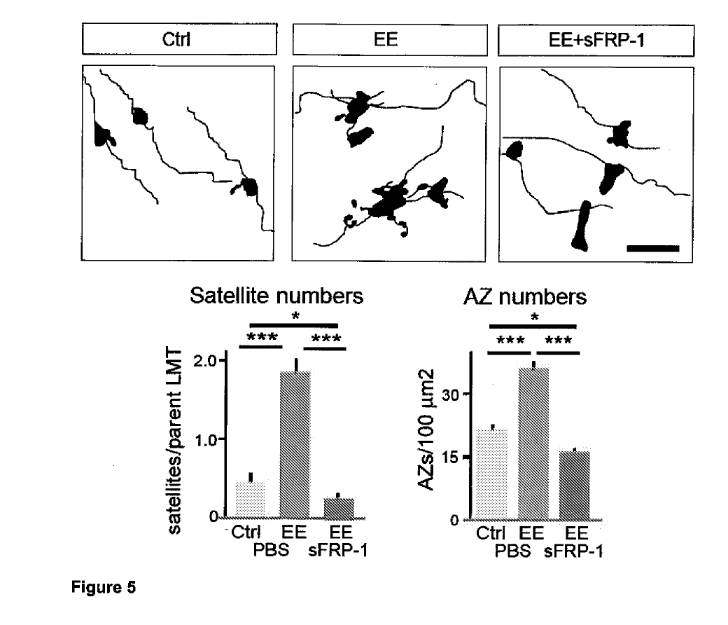

FIG. 6: Wnt signaling is required to enhance synapse and LMT numbers in the presence or absence of Rab3a. sFRP-1 also effectively suppressed the effect of EE on LMT numbers and complexities in Rab3a^{-/-} mice.

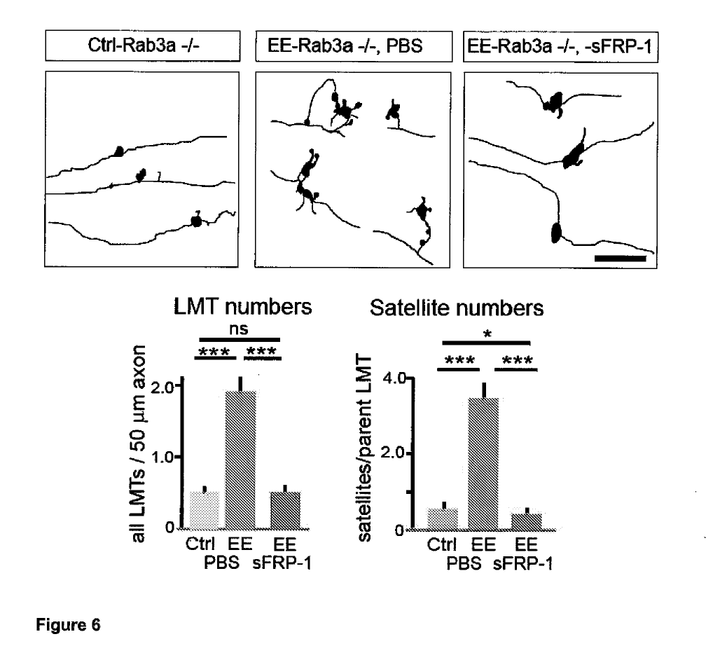

FIG. 7: Wnt agonist produces effects closely comparable to those of the EE conditions. To determine whether enhanced Wnt signaling might be sufficient to enhance stratum lucidum synapse numbers and LMT complexities, the inventors applied a cell permeable, potent and selective agonist of Wnt signaling locally into CA3 in mice housed under control conditions. It was found that the Wnt agonist produced effects closely comparable to those of the EE conditions, including increased stratum lucidum LMT numbers, stratum lucidum AZ numbers, and AZ densities at LMTs. The Wnt agonist also increases LMT satellite numbers when applied to organotypic slice cultures (not shown). Taken together, these results provided evidence that Wnt signaling regulates synapse numbers and LMT complexities in adult mice under control and EE conditions.

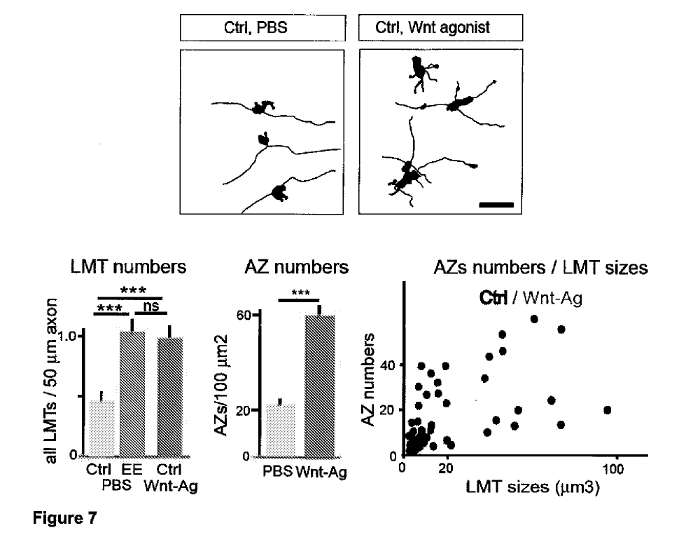

FIG. 8: Application of sFRP-1 locally into CA3 in mice housed under control conditions produces a reduction in total stratum lucidum AZ numbers and in the contents of AZs per LMT. To investigate the possibility that stratum lucidum synapse numbers in the adult might undergo constant regulation mediated by Wnt signaling, sFRP-1 was applied locally into CA3 in mice housed under control conditions. This treatment produces a reduction in total stratum lucidum AZ numbers, in the contents of AZs per LMT, and in the fractions of LMTs with satellites (not shown).

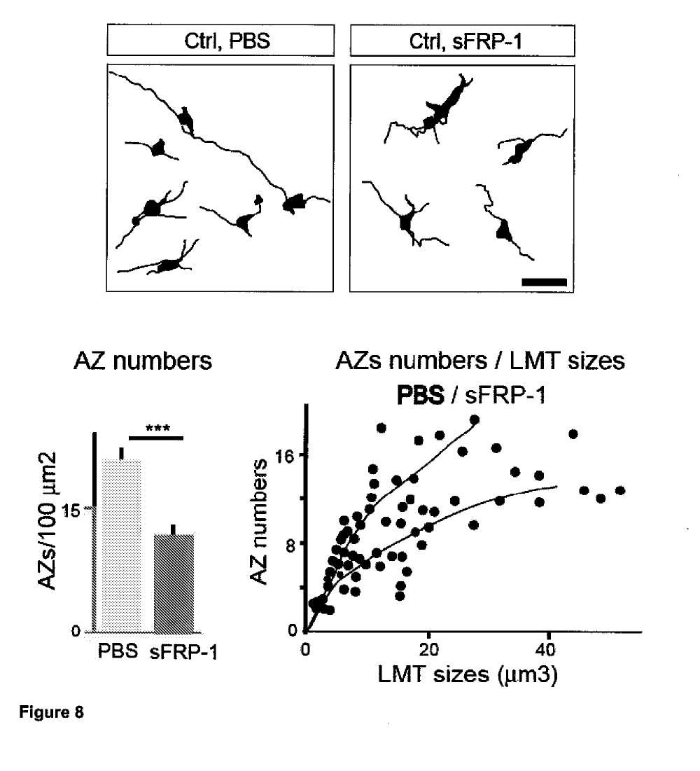

### DETAILED DESCRIPTION OF THE INVENTION

The present inventors have now surprisingly found that EE specifically and reversibly produces a robust increase in stratum lucidum synapse numbers, and in the densities of synapses per LMT. In parallel, the EE conditions produce a marked increase in CA3 pyramidal neuron Wnt7a/b protein levels, a member of the Wnt-signaling pathway. Local inhibition of Wnt signaling in CA3 suppresses the effects of EE on synapse numbers, and further reduces synapse numbers in mice housed under control conditions; conversely, the local application of a Wnt-signaling agonist is sufficient to mimick the effects of EE, both in vivo and in slice cultures. Taken together, these surprising results show that Wnt signaling mediates global regulation of synapse numbers in response to experience and age, not only in young animals, i.e. before and during the normal developmental synapse elimination and critical periods, but also in adult hippocampal stratum lucidum. These sustained global alterations in synapse numbers and connectivities hence represent a novel class of structural plasticity mechanisms, which allow to modify network properties in response to experience.

The present invention hence provides a method of preventing and/or treating a disease characterized by a loss or imbalance of neuronal connections in a subject by administering to said subject a therapeutically effective amount of a modulator of Wnt-signaling, wherein (i) said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, and wherein (ii) said subject is in puberty or post-puberty, wherein puberty or post-puberty means that the neuronal circuits of said subject have already undergone developmental synapse elimination and critical periods which take place prior to puberty.

Another embodiment of the invention encompasses a method of modulating neuronal activity and/or neuronal plasticity, optimizing repair after brain injury, modulating learning, and/or modulating cognitive capabilities in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty. A further embodiment of the present invention is a method of preventing loss of neuronal connections and/or increasing neuronal connections in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty.

In the methods of the invention, Wnt-signaling can be modulated by an agonist of Wnt-signaling, for instance a small molecule, and antibody or a siRNA.

In some embodiments of the invention, the subject is a mammal, for example a human subject. Moreover, in some embodiments, the modulation of the Wnt-signaling can occur in the hippocampus of the subject.

In a particular embodiment of the invention, the expression and/or biological activity of Wnt7a and/or Wnt7b is modulated, for instance increased.

In a further aspect, the present invention encompasses a siRNA decreasing or silencing the expression of an antagonistic component of Wnt-signaling, thus for example increasing the level of Wnt7a and/or Wnt7b, for use as a medicament in a subject who is in puberty or post-puberty, wherein puberty or post-puberty means that the neuronal circuits of said subject have already undergone developmental synapse elimination and critical periods which take place prior to puberty, to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject.

Moreover, the present invention also encompasses an antibody specifically binding to a modulator of Wnt-signaling, for instance an antagonist of Wnt-signalling, for use as a medicament in a subject who is in puberty or post-puberty, wherein puberty or post-puberty means that the neuronal circuits of said subject have already undergone developmental synapse elimination and critical periods which take place prior to puberty to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject. For instance, the antibody of the invention can inhibit the binding of said Wnt-signaling component to its ligand. In some embodiment, the antibody acts as an agonist of Wnt-signalling by acting in an agonistic way on its specific target.

The present invention also encompasses a drug delivery device for implantation into the nervous system of a subject who is in puberty or post-puberty, wherein puberty or post-puberty means that the neuronal circuits of said subject have already undergone developmental synapse elimination and critical periods which take place prior to puberty, the drug delivery device comprising: a biocompatible polymer; and a modulator of Wnt-signaling, for instance an agonist, wherein said modulator of Wnt-signaling is released from the polymer into the nervous system of the subject in an amount effective to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorder's, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject.

These and other aspects of the present invention should be apparent to those skilled in the art, from the teachings herein.

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g. , a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single-and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al. , Meth Enzymol 182:626-46 (1990); Rattan et al., Ann NY Acad Sci 663:48-52 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence aligmnent, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty—1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs.

A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=1, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level.

Alternatively, non-naturally occurring variants may be produced by mutagenesis techniques or by direct synthesis. Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N-or C-terminal residues of a protein retains such immunogenic activities can readily be determined by routine methods described herein and otherwise known in the art.

In one embodiment where one is assaying for the ability to bind or compete with full-length Frizzled polypeptide for binding to anti-Frizzled antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffasion assays, in situ immunoassays (using colloidal gold, enzyme or radioisotope labels, for example), western blots, precipitation reactions, agglutination assays (e.g., gel agglutination, assays, hemagglutination assays), complement fixation assays, immunofluorescence assays, protein A assays, and immunoelectrophoresis assays, etc. In one embodiment, antibody binding is detected by detecting a label on the primary antibody.

In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. Assays described herein and otherwise known in the art may routinely be applied to measure Wnt-signaling, for instance, the ability of Frizzled polypeptides and fragments, variants derivatives and analogs thereof to elicit Frizzled-related biological activity (either in vitro or in vivo). For example, 6-catenin/TCF dependent reporter gene in a Luciferase assay is commonly used.

The term “epitopes,” as used herein, refers to portions of a polypeptide having antigenic or immunogenic activity in an animal, in some embodiments, a mammal,for instance in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)).

The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CH1, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e. g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgAl and IgA2) or subclass of immunoglobulin molecule.

In addition, in the context of the present invention, the term “antibody” shall also encompass alternative molecules having the same function and/or properties, e.g. aptamers and/or CDRs grafted onto alternative peptidic or non-peptidic frames.

In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. lmmunol. 147:60-59 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%. less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognised polypeptides. The invention also features receptor-specific antibodies which do not prevent ligand binding but prevent receptor activation. Receptor activation (i.e., signalling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387.

The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al. , Antibodies: A Laboratory Manual, (Cold Spring Harbor Laboratory Press, 2nd ed. 1988); Hammerling, et al., in: Monoclonal Antibodies and T-Cell Hybridomas 563-581 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain. For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immumol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity.

Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-08 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies. Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide).

Further, an antibody or fragment thereof may be conjugated to a therapeutic moiety. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et aL, Int. Immunol., 6:1567-1574 (1994)), VEGI (See, International Publication No. WO 99/23105) , a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat cancer.

Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

The invention also provides methods for treating a subject by increasing Wnt-signaling by administration to the subject of an effective amount of an agonistic compound or pharmaceutical composition comprising such a compound. In some embodiments, said agonistic compound is an antibody or an siRNA. In a preferred aspect, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human. Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. , Goodson, in Medical Applications of Controlled Release, supra, vol. 2, pp. 115-138 (1984)).

Other controlled release systems are discussed in the review by Langer (Science 249:1527-1533 (1990)). The present invention also provides pharmaceutical compositions for use in the methods of the invention by modulating Wnt-signaling. Such compositions comprise a therapeutically effective amount of an modulatory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, driied skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection. Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems.

For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for examplel mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like. The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-546 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown’ of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustaIX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)* 100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-55° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid.

The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable.

Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs. washington.edu/phylip.html) using the “Similarity Table” option as the model for amino acid substitution (P). For DNA/RNA, an identical option exists within the DNADIST program of PHYL1P.

The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the modulator is a siRNA molecule and comprises between approximately 5 bp and 50 bp, in some embodiments, between 10 by and 35 bp, or between 15 by and 30 bp, for instance between 18 by and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among:
a) a 3′ cap;
b) a 5′ cap,
c) a modified internucleoside linkage; or
d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132.

Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi. It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators). The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible. Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like). Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In a preferred embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

Modulators according to any embodiment of the present invention may be used in a monotherapy (e.g. use of siRNAs alone). However it will be appreciated that the modulators may be used as an adjunct, or in combination with other therapies.

The modulator of Wnt-signaling may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the modulator to the target site.

The modulator of Wnt-signaling according to the present invention may be used in a number of ways. For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The modulators may also be administered by inhalation (e.g. intranasally) or even orally (if appropriate).

The modulators of the invention may also be incorporated within a slow or delayed release device. Such devices may, for example, be inserted at the site of a tumour, and the molecule may be released over weeks or months. Such devices may be particularly advantageous when long term treatment with a modulator of Wnt-signaling is required and which would normally require frequent administration (e.g. at least daily injection).

It will be appreciated that the amount of an modulator that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the modulator within the subject being treated.

Optimal dosages to be administered may be determined by those skilled in the art, and will vary with the particular modulator in use, the strength of the preparation, and the mode of administration.

Additional factors depending on the particular subject being treated will result in a need to adjust dosages, including subject age, weight, gender, diet, and time of administration.

When the modulator is a nucleic acid conventional molecular biology techniques (vector transfer, liposome transfer, ballistic bombardment etc) may be used to deliver the modulator to the target tissue.

Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of a modulator of Wnt-signaling may be used for the treatment of cancer in the subject, depending upon which specific modulator is used. When the modulator is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the modulator (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

As used herein, the term “Wnt-signaling” refers to the Wnt signaling pathway . The Wnt signaling pathway describes a complex network of proteins most well known for their roles in embryogenesis and cancer, but also involved in normal physiological processes in adult animals. Examples of components of the Wnt-signaling in human are WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, and WNT16. The Wnt-signaling pathway involves a large number of proteins that can regulate the production of Wnt signaling molecules, their interactions with receptors on target cells and the physiological responses of target cells that result from the exposure of cells to the extracellular Wnt ligands. Although the presence and strength of any given effect depends on the Wnt ligand, cell type, and organism, some components of the signaling pathway are remarkably conserved in a wide variety of organisms, from Caenorhabditis elegans to humans. Protein homology suggests that several distinct Wnt ligands were present in the common ancestor of all bilaterian life, and certain aspects of Wnt signaling are present in sponges and even in slime molds. The canonical Wnt pathway describes a series of events that occur when Wnt proteins bind to cell-surface receptors of the Frizzled family, causing the receptors to activate Dishevelled family proteins and ultimately resulting in a change in the amount of β-catenin that reaches the nucleus. Dishevelled (DSH) is a key component of a membrane-associated Wnt receptor complex which, when activated by Wnt binding, inhibits a second complex of proteins that includes axin, GSK-3, and the protein APC. The axin/GSK-3/APC complex normally promotes the proteolytic degradation of the β-catenin intracellular signaling molecule. After this “β-catenin destruction complex” is inhibited, a pool of cytoplasmic β-catenin stabilizes, and some β-catenin is able to enter the nucleus and interact with TCF/LEF family transcription factors to promote specific gene expression.

Cell surface Frizzled (FRZ) proteins usually interact with a transmembrane protein called LRP. LRP binds Frizzled, Wnt and axin and may stabilize a Wnt/Frizzled/LRP/Discheveled/axin complex at the cell surface (“receptor complex”).

In vertebrates, several secreted proteins have been described that can modulate Wnt signaling by either binding to Wnts or binding to a Wnt receptor protein. For example, Sclerostin can bind to LRP and inhibit Wnt signaling. Several protein kinases and protein phosphatases have been associated with the ability of the cell surface Wnt-activated Wnt receptor complex to bind axin and disassemble the axin/GSK3 complex. Phosphorylation of the cytoplasmic domain of LRP by CK1 and GSK3 can regulate axin binding to LRP. The protein kinase activity of GSK3 appears to be important for both the formation of the mebrane-associated Wnt/FRZ/LRP/DSH/Axin complex and the function of the Axin/APC/GSK3/β-catenin complex. Phosphorylation of β-catenin by GSK3 leads to the destruction of β-catenin.

“WNT7A”, or Wingless-type MMTV integration site family, member 7A is a human gene. This gene is a member of the WNT gene family. It encodes a protein which shows 99% amino acid identity to the mouse Wnt7A protein. This gene not only guides the development of the anterior-posterior axis in the female reproductive tract, but also plays a critical role in uterine smooth muscle pattering and maintenance of adult uterine function. It is also responsive to changes in the levels of sex steroid hormone in the female reproductive tract. Decreased expression of this gene in human uterine leiomyoma is found to be inversely associated with the expression of estrogen receptor-alpha.

“WNT7B”, or Wingless-type MMTV integration site family, member 7B, is also a human gene. This gene is a member of the WNT gene family. It encodes a protein which shows 99% and 91% amino acid identity to the mouse and Xenopus Wnt7A proteins, respectively. Among members of the human WNT family, this protein is most similar to WNT7A protein (77.1% total amino acid identity). This gene may play important roles in the development and progression of gastric cancer, esophageal cancer, and pancreatic cancer.

The term “Frizzled” refers to a family of G protein-coupled receptor proteins, having 7-transmembrane domain, that serve as receptors in the Wnt signaling pathway and other signaling pathways. Most frizzled receptors are coupled to the beta-catenin canonical signaling pathway. When activated, Frizzled leads to activation of Dishevelled in the cytosol. Frizzled proteins and the genes that encode them have been identified in an array of animals, from sponges to humans. Frizzled proteins also play key roles in governing cell polarity, embryonic development, formation of neural synapses, cell proliferation, and many other processes in developing and adult organisms. “Frizzled-1” or Frizzled homolog 1 (Drosophila), also known as FZD1, is a human member of the frizzled gene family. The FZD1 protein contains a signal peptide, a cysteine-rich domain in the N-terminal extracellular region, 7 transmembrane domains, and a C-terminal PDZ domain-binding motif. The FZD1 transcript is expressed in various tissues. The Entrez GenelD for Frizzled-1 is: 8321. “Frizzled-4” or Frizzled homolog 4 (Drosophila), also known as FZD4, is also a human member of the frizzled gene family. This protein may play a role as a positive regulator of the Wingless type MMTV integration site signaling pathway. A transcript variant retaining intronic sequence and encoding a shorter isoform has been described, however, its expression is not supported by other experimental evidence. Mutations in the human frizzled-4 receptor have been linked to familial exudative vitreoretinopathy, a rare disease affecting the retina at the back of the eye, and the vitreous, the clear fluid inside the eye. The Entrez GeneID for Frizzled-4 is: 8322.

“Modulators of Wnt-signaling” are known and include, e.g. secreted proteins that have been described to modulate Wnt signaling by either binding to Wnts or binding to a Wnt receptor protein. For example, Sclerostin can bind to LRP and inhibit Wnt signaling. Small molecules capable of modulating Wnt-signaling are also known. For example, 2-amino-4-[3,4-(methylenedioxy)benzyl-amino]-5-(3-methoxyphenyl)pyrimidine acts as an agonist of Wnt signaling. Further examples of Wnt-signaling modulators can be found in the art, e.g. in WO/2007/074346 or in WO/2006/116503. The modulators of the present invention include those that directly modulate the wnt signaling pathway; modulate the prostaglandin pathway to effect modulation of the wnt signaling pathway; or modulate the downstream effects of prostaglandin to modulate wnt. Additionally, these modulators may be combined to “fine tune” the signal. For example, a wnt signaling activator may be employed until the desired enhancement is noted, followed by a prostaglandin modulator to limit the effects of the wnt activator. Or, for example, a low-dose wnt activator could be combined with a low-dose prostaglandin activator to avoid toxicity. Thus, the interactions of the modulators of the wnt and prostaglandin signaling pathways may be used in any direction or in any combination to elicit a desired response while limiting toxicity or exuberant growth. The modulators may be used simultaneously or sequentially.

As used herein, the term “agonist” generally refers to a substance that can directly interact with (e.g., bind to) a receptor and initiate a physiological or a pharmacological response characteristic of the activity of that receptor, e.g., the activity that is normally induced by interaction of an endogenous positively-acting ligand with the receptor. Substances generally recognized in the literature as agonists of Wnt-signaling are of use in the methods of the invention. The term “agonist” also refers to partial agonists, i.e., compounds that are capable of partially activating Wnt-signaling, e.g., activating it to a lesser extent than its endogenous ligand. The term also encompasses substances that indirectly stimulate a receptor, e.g., by inhibiting reuptake or breakdown/metabolism of an endogenous direct agonist and/or by stimulating the production or release of an endogenous direct agonist.

As used herein, the term “antagonist” generally refers to a substance that opposes the Wnt-signaling-associated responses normally induced by another bioactive agent such as an endogenous positively-acting ligand. Typically, an antagonist binds to a receptor and prevents binding of an endogenous ligand that would normally activate the receptor, or prevents binding of an exogenous agonist to the receptor. The antagonist may or may not induce an effect itself. The activity of a receptor is generally taken to be the activity associated with binding of an endogenous positively-acting ligand. Substances generally recognized in the literature as antagonists of a particular receptor are of use in the methods described herein. The term also encompasses substances that indirectly inhibit a receptor, e.g., by inhibiting reuptake or by stimulating breakdown/metabolism of an endogenous direct agonist and/or by stimulating the production or release of an endogenous direct antagonist.

A material is considered “biocompatible” if it is substantially non-toxic to the recipient, in the quantities and at the location used, and also does not elicit or cause a significant deleterious or untoward effect on the recipient's body, e.g., a significant immunological or inflammatory reaction, unacceptable scar tissue formation, etc.

As used herein, the term “biodegradable,” refers to a material that is capable of being broken down physically and/or chemically within the body of a subject, e.g. , by hydrolysis under physiological conditions, by natural biological processes such as the action of enzymes present within the body, etc., to form smaller chemical species which can be metabolized and/or excreted.

The “central nervous system” (CNS) includes the brain, spinal cord, optic, olfactory, and auditory systems. The CNS comprises both neurons and glial cells (neuroglia), which are support cells that aid the function of neurons. Oligodendrocytes, astrocytes, and microglia are glial cells within the CNS. Oligodendrocytes myelinate axons in the CNS, while astrocytes contribute to the blood-brain barrier, which separates the CNS from blood proteins and cells, and perform a number of supportive functions for neurons. Microglial cells serve immune system functions.

The term “concurrent administration,” as used herein with respect to two or more agents, e.g., therapeutic agents, is administration performed using doses and time intervals such that the administered agents are present together within the body, or at a site of action in the body such as in the CNS in amounts sufficient to have a biological effect over a time interval of minutes, hours, days, weeks, etc. The agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart).

As used herein, the term “critical period” refers to a time period during the development of an organism in which the organism's nervous system is particularly able to acquire a specific functional ability and/or structural configuration, typically at least in part in response to external environmental stimuli. Absence of the appropriate stimuli during the critical period typically results in failure to develop the functional ability and/or structural configuration that would develop had these stimuli been present. The timing and duration of the critical period may depend upon the environmental stimuli received. For example, lack of certain environmental stimuli prolongs the critical period.

As used herein, the term “deprived condition” refers to an environment that fail to provide adequate environmental stimuli needed to allow normal development of one or more functional or structural features of the nervous system. An individual subjected to a deprivation condition typically receives fewer and/or less intense or varied stimuli of one or more types than an individual subjected to “normal conditions.” In the case of an animal raised in a laboratory, “normal conditions” are standard laboratory conditions typically used for the maintenance of such animals.

As used herein, an “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. A desired biological response may be, for example, (i) functional or structural reorganization of synaptic connections, dendrites, or axon projections; (ii) maintenance of synaptic connections, dendrites, or axon projections under conditions in which they would otherwise deteriorate ; (iii) regeneration of a nerve or an axonal projection system or its maintenance under conditions in which it would otherwise deteriorate; (iv) an improvement in performance of a task requiring motor or sensory function; (v) an improvement in performance of a task requiring cognitive function, e.g., improved performance on a test that measures learning and/or memory; (vi) a slowing in the rate of decline in motor, sensory, and/or cognitive function.

As used herein, the term “enriched condition” refers to an environment that provides receives more stimuli and/or more intense or varied stimuli of one or more types than an individual subjected to “normal conditions.”

As used herein, the term “expression product” or “gene product” refers to an RNA transcribed from a gene or a polypeptide translated from an RNA transcribed from a gene. RNAs or polypeptides that are modified following their transcription or translation are considered expression products of the gene that encodes them. Modifications include, e.g., splicing, cleavage, addition of phosphate or fatty acid groups, etc.

As used herein, the term “focal delivery” (or “focal administration” in reference to delivery of a pharmacological agent), refers to delivery that does not rely upon transport of the agent to its intended target tissue via the vascular system, e.g., the agent is not administered directly into a blood vessel. The agent is delivered directly to its intended target tissue or in the vicinity thereof, e.g. by injection through a needle, catheter, or cannula, or by implantation of a delivery vehicle or device containing the agent. If the agent is delivered to the vicinity of its target tissue rather than into the target tissue itself, the agent may reach its target tissue by diffusion. For purposes of the present invention, any method that achieves delivery of an agent to the CNS or portion thereof without requiring transport via the vascular system from a site outside the skull or meninges (the membranes that cover the brain and the spinal cord), is considered to achieve focal delivery of the agent. Specifically included are delivery by use of an implanted or external pump, and/or delivery directly into one or more ventricles of the CNS. It will be understood that once having been focally delivered a portion of the agent (typically only a minor fraction thereof) may in part enter the vascular system and be transported to another location.

As used herein, the term “function,” with reference to the nervous system or a component thereof, is used broadly herein to refer to any function, role, task, or activity performed by the nervous system or a component thereof. The term includes, without limitation, the ability to process and recall information, regulate behavior, stimulate release of endogenous chemicals, control motor functions, receive and process sensory input, maintain consciousness, etc.

As used herein, the term “functional recovery” refers to the process in which a nervous system or component thereof that has at least in part lost the ability to perform a function that it previously performed, regains at least in part the ability to perform the function. Functional recovery may take place in at least two different ways: (i) the recovery in function may involve partial or complete recovery of the portion of the nervous system that previously performed the function; (ii) the recovery in function may involve a portion of the nervous system performing a function that it did not previously perform. Of course in some instances both processes may take place. Functional recovery can also refer to preservation of the ability of the nervous system or a portion thereof to perform a function that it previously performed, after the nervous system or component thereof has been physically altered, disrupted, or otherwise subjected to a physical or chemical insult or neurodegenerative disease, when such physical alteration, disruption, physical or chemical insult or neurodegenerative disease would otherwise be expected to lead to deterioration or loss of the ability of the nervous system or portion thereof to perform the function.

The term “functional reorganization,” as used in reference to the nervous system or a portion thereof, refers to the process in which a portion of the nervous system wholly or partially assumes, i.e., takes on, a function (e.g., a sensory, motor, or cognitive function) that was not previously performed by that portion of the nervous system. The function or task may, but need not have been, previously performed by a different portion of the nervous system. Functional reorganization may, but need not, entail one or more aspects of structural reorganization. Functional reorganization may also be referred to as functional rearrangement. An example of functional reorganization is the capacity of an area of sensory or motor cortex adjacent to an area of injury or necrosis of CNS tissue to control CNS output to a portion of the body that was previously controlled by the injured or necrotic tissue, or to receive and process input from a region of the body from which input was previously received and processed by the injured or necrotic tissue. Another example is the capacity of an area of sensory or motor cortex corresponding in location to an area of injury or necrosis of CNS tissue, but located in the opposite hemisphere of the brain, to control CNS output to a portion of the body that was previously controlled by the injured or necrotic tissue, or to receive and process input from a region of the body from which input was previously received and processed by the injured or necrotic tissue. Yet another example is provided by the nervous system's response to monocular deprivation.

As used herein, the term “infarct” refers to an area of localized tissue necrosis resulting from inadequate blood supply, e.g., due to obstruction of a blood vessel. Also referred to as an infarction. When the necrotic tissue is brain tissue, the infarct may be referred to as a cerebral infarct or cerebral infarction.

As used herein, the term “modulate” means to alter, e.g., to increase or enhance, to decrease or inhibit, or to cause a variation in a temporal pattern. To “modulate a gene” means to modulate the level and/or activity of an RNA or polypeptide expression product of the gene, e.g., by administering an agonist or antagonist. “Level” of an expression product refers to amount, e.g., concentration by weight or volume, number of molecules per cell or by weight or volume, etc. To “modulate a pathway” means to modulate at least one reaction and/or gene involved in the pathway, typically resulting in an alteration in the biological effect or outcome of the pathway. To “modulate a cell” means to increase or enhance, or to decrease or inhibit, the development, survival, and/or activity of the cell.

As used herein, the term “neural tissue” refers to one or more components of the central nervous system and/or peripheral nervous system. Such components include brain tissue and nerves, which may be present in bundles or tracts. In general, brain tissue and nerves contain neurons (which typically comprise cell body, axon, and dendrite(s)), glial cells {e.g., astrocytes, oligodendrocytes, and microglia in the CNS; Schwann cells in the PNS). It will be appreciated that brain tissue and nerves typically also contain various noncellular supporting materials such as basal lamina (in the PNS), endoneurium, perineurium, and epineuriun in nerves, etc. Additional nonneural cells such as fibroblasts, endothelial cells, macrophages, etc., are typically also present. See Schmidt and Leach, 2003, for further description of the structure of various neural tissues.

As used herein, the term “peripheral nervous system” (PNS) includes the cranial nerves arising from the brain (other than the optic and olfactory nerves), the spinal nerves arising from the spinal cord, sensory nerve cell bodies, and their processes, i.e., all nervous tissue outside of the CNS. The PNS comprises both neurons and glial cells (neuroglia), which are support cells that aid the function of neurons. Glial cells within the PNS are known as Schwann cells, and serve to myelinate axons by providing a sheath that surrounds the axons. In various embodiments of the invention the methods and compositions described herein are applied to different portions of the PNS.

As used herein, the term “plasticity” refers to the capacity of the nervous system, or a portion thereof, to change (e.g., to reorganize) its structure and/or function, generally in response to an environmental condition, injury, experience, or ongoing nervous system activity. Plasticity may involve the proliferation of neurons or glia, the growth or movement of neuronal processes and/or alterations in their shape. Plasticity may involve formation of new synaptic connections between neurons and/or strengthening or weakening of existing synaptic connections. Formation of new synaptic connections may involve growth or movement of neuronal processes. Plasticity may also involve alterations in non-neuronal components of the nervous system, e.g., astrocytes or other glial cells. As used herein, the term “synapses” refer to “specialized intercellular junctions between neurons or between neurons and other excitable cells where signals are propagated from one cell to another with high spatial precision and speed” (De Camilli, in Cowan, supra). They are the primary sites of intercellular communication in the mammalian nervous system. In general, the basic structure of a synapse consists of a close juxtaposition of specialized regions of the plasma membrane of two neurons, referred to as the presynaptic and postsynaptic neurons, to form a synaptic junction. The presynaptic neuron is the nerve cell transmitting a signal while the postsynaptic neuron is the recipient of the signal. Most neurons in the vertebrate nervous system possess a cell body and two types of cell processes, axons and dendrites. Signals, i.e., action potentials, are initiated and transmitted by the axon while dendrites (and also the cell body) receive inputs via synaptic contacts from other neurons.

As used herein, the term “treating” generally refers to medical and/or surgical management of a patient for purposes of bringing about an improvement in the state of a subject with respect to a disease, disorder, or condition from which the subject suffers and/or reducing or slowing further deterioration of the subject's condition. Treating can include reversing, alleviating, and/or inhibiting the progress of, the disease, disorder, or condition to which such term applies, and/or reversing, alleviating, inhibiting the progress one or more symptoms or manifestations of such disease, disorder or condition.

The term “psychiatric disorders” includes, but is not limited to, depression, obsessive compulsive disorder (OCD), generalized anxiety disorder, anorexia nervosa, social anxiety disorder, bulimia nervosa, Tourette syndrome, asperger's syndrome, compulsive skin picking, body dysmorphic disorder, trichotillomania, obsessive-compulsive personality disorder, schizophrenia, visual hallucination, auditory hallucination, eating disorder, and bipolar disorder.

The present invention also provides a method of screening compounds to identify those which might be useful for performing the methods of the invention by modulating Wnt-signaling, as well as the so-identified compounds. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### EXAMPLES

Mice and Reagents

Transgenic mice expressing membrane-targeted GFP in only a few neurons (Thy1-mGFPs, L21 and L15) were as described (De Paola et al., 2003). Transgenic males were crossed over more than ten generations with non-transgenic F1 offsprings from C57BI6×BalbC crosses, so that the genetic background of the mice was 50% each of C57BI6 and BalbC. Rab3a knockout mice (B6; 129S-Rab3a^{tm1Sud}/J) were obtained from the Jackson Laboratory and backcrossed into Thy1-mGFP (L21) mice.

For enriched environment (EE) experiments, male mice were housed in groups of 3-4 littermates in large (rat) cages equipped with 3 running wheels per cage, toys and hiding spaces. Control mice were male littermates (3-4), which were housed individually in small standard cages without special equipment. Unless stated otherwise, EE was started at 1 month of age, and lasted 20 days.

Organotypic slice cultures were based on the Stoppini method (Stoppini et al., 1991), as described (De Paola et al., 2003).

Mature recombinant sFRP-1 was obtained from R&D systems and dissolved in PBS/0.1% BSA (vehicle: PBS/0.1% BSA) to a working concentration of 50 μg/ml. Wnt agonist (Calbiochem) was dissolved in a small volume of DMSO and then further diluted with PBS to a final concentration of 10 mM (DMSO<1:1000). PBS/DMSO solution alone was used as vehicle for control injections. Antibodies were from the following sources: Bassoon, anti-mouse Alexa Fluor 546 and 647, anti-goat Alexa Fluor 546 and 633, donkey anti-goat Alexa Fluor 568 (Molecular Probes); NeuN anti-mouse (CHEMICON); anti-human Wnt7a/b antibody (R&D, P56706). The Wnt7a/b antibody preferentially detects Wnt7b (99% identity between mature mouse and human Wnt7b), but exhibits ca. 20% cross-reactivity for Wnt7a (R&D); given that the two Wnt proteins are very similar structurally and functionally (80% sequence identity), in the present experiments, the inventors made no attempts to distinguish them in their experiments, and designated them as Wnt7a/b. Reagents, with their working concentrations were: TTX (Laxotan, 1 μM, stock in acetate buffer), Bicuculine (SIGMA, 20 μM in water), CGP (SIGMA, 0.4 μM in water), Picrotoxin (SIGMA, 20 μM in water).

Surgery and In Vivo Treatments

Injection canulas were implanted unilaterally (left hemisphere) above the CA3 b-a region of Isofluorane-anesthetized mice using stereotactical guidance. Canulas were fixed with dental cement. Implantation coordinates from Bregma (implantation 0.5 mm above injection site as the needle surpassed canula by 0.5 mm): ante-posterior −2.06 mm, lateral 2.5 mm, dorso-ventral −1.4 mm. Animals were allowed to recover for 3 days after surgery before drug treatments started.

Animals were injected under Isofluorane anesthesia with 300 nl of either drug (treated) or vehicle (control) through a small needle guided through the implanted canulas every 3-4 days for 12-21 days (depending on experiment). Canula positions were confirmed in all analyzed sections to be above Ca3b-a without harming the mossy fiber projection or CA3 pyramidal cell dendrites.

Immunohistochemistry and Histology

Vibratome sections were of 60 μm thickness. Free-floating sections were then post-fixed with 50% Ethanol in PBS for 15-20 minutes, and subsequently permeabilized/blocked for 3 hours at RT in 20% BSA/0.5% TritonX100 in PBS. Sections were then incubated with primary, and subsequently secondary antibody (3-4 hours at RT each in 5% BSA in PBS). Between and after antibody incubations, sections were washed 3×10 minutes in 5% BSA in PBS on a shaker. Sections were then mounted in ProLong Gold (Molecular Probes) and imaged.

For electron microscopy, mice were perfused with buffered 2.5% glutaraldehyde, followed by fixation in buffered 2.5% glutaraldehyde (2 h), post-fixation in buffered 2% Osmium tetroxide (2 h), and dehydration through alcohol, followed by propylene oxide. Fixed brain material (hippocampal CA3a) was embedded in Docupon, stained with uranyl acetate and lead hydroxide, and sectioned with a diamond knife. Sections were recorded on Kodak electron image plates using a Zizze EM900 at 100 kV. For quantitative analysis, thorns were defined as rounded profiles at least partially included into LMTs. Synapses were identified by the presence of characteristic postsynaptic densities, by a local widening of the extracellular space. For synapse densities along pre-/post-contacts, we divided the sum of all contact lengths between thorns and LMTs on individual electron micrographs (ImageJ), by the sum of all corresponding synapses.

Image Acquisition and Analysis

For imaging of LMTs from mice of different ages, genotypes and life-styles, male mice were perfused transcardially with 100 ml ice-chilled 4% paraformaldehyde in PBS; brains were collected and kept in fixation solution over-night at 4° C. Vibratome coronal sections (100-150 μm) were cut the next day using a LEICA VT 100S vibratome (Leica), and mounted in Airvol for fluorescence imaging. High resolution images were acquired on an upright spinning disc confocal microscope consisting of a Yokogawa CSU22 confocal scanning head mounted on a Zeiss Axioimager M1 using a 100× alphaPlan-Apochromat 1.45 (Zeiss) oil immersion objective.

Immunocytochemistry preparations were imaged on an LSM510 confocal microscope (Zeiss) using a 40× (1.4) oil-immersion objective. Imaging of organotypic slice cultures was as described. Briefly, images were acquired with an LSM 510 (Zeiss), using a 40× (1.4) oil objective (Zeiss); image sizes were 2048×2048, and stepsizes were 0.4 μm. Where indicated, drugs were replaced every 3-4 days, together with fresh medium.

Microscope images were processed and analysed using Metamorph 6.1, Imaris 4.2 (Bitplane AG) and ImageJ softwares. We defined LMTs as mossy fiber terminal regions of >2.5 μm diameter in CA3a-c, which were arranged either en-passant, or as side structures connected to the mossy fiber axon or another LMT by an axonal process (satellite). For the quantification of LMT sizes in 3D (volumes) at least three confocal 3D stacks were acquired in CA3b for each preparation (three mice per condition), and analyzed using Imaris 4.2 software.

Individual LMT volumes contained in these cubes were measured using the Surpass/ Isosurface function of the software. Non-saturating imaging conditions were chosen for all size analyses. An intensity threshold of 300 was chosen to selectively analyze LMTs (excluding axons and other smaller objects). All identified objects were verified by eye inspection. Generally, the settings for the analysis were identical for all samples of all ages, but in some cases we verified the settings through internal calibration using the diameters and signal intensities of axons. After these measurements, LMTs were grouped according to their volumes. The volumes of all LMTs in one group were added up, and expressed as percentage of the total volume of all LMTs measured per cube. For the quantification of LMT sizes in 2D, MIPs of 3D stacks were created in ImageJ, and LMT surfaces (areas in μm²) were measured. For all samples, LMTs were inspected by eye in 3D to be identified as individual LMTs and not fused with other LMTs. LMT numbers were determined in CA3b as follows: the sum of all mGFP-positive LMTs within a region of interest (ROI; a MIP) was divided by the number of mGFP-labeled axons within that ROI, and by the length along CA3 included in the ROI. LMT numbers were then normalized to 100 μm axon length. Wnt7a/b staining intensities were measured on single confocal planes. Small ROls (˜2-3 μm²) were created using ImageJ software, and the mean intensity of grey values was measured inside these ROIs placed within pyramidal cell bodies, or inside and outside debdritic clusters. Stainings within one series were performed in parallel, and imaging settings were adjusted to be optimal for the entire set of samples belonging to one experiment. These settings were then kept for the acquisition of all images within one experiment. Wnt7a/b positive clusters were defined as distinct accumulations larger than 4 μm²; all clusters were counted within ROls within CA3a-b. For the quantification of long/short axis ratios, thin MIPs were created in Metamorph, and the longest extension=long axis (perpendicular to mossy fiber axon) was measured using ImageJ software. For the length of the short axis, the longest extension of the same LMT perpendicular to the first measurement was taken. For the quantitative analysis of AZs in stratum lucidum, all Bassoon-positive puncta per 100 μm² ROI in CA3b were counted on single confocal sections. For the quantitative analysis of Bassoon-positive puncta per individual LMT, LMT volumes were first determined in 3D as described above. Confocal stacks were then opened individually, and for any given LMT Bassoon spots overlapping with the GFP signal were counted on consecutive optical sections, throughout the entire z-extension of the terminal. Puncta that were continuous on consecutive sections were excluded. For expression of Bassoon densities per LMT volume we divided the number of counted Bassoon spots per LMT by it's measured volume.

### REFERENCES

Abe, K., and Takeichi, M. (2007). NMDA-receptor activation induces calpain-mediated beta-catenin cleavages for triggering gene expression. Neuron 53, 387-397.

Acsady, L., Kamondi, A., Sik, A., Freund, T., and Buzsaki, G. (1998). GABAergic cells are the major postsynaptic targets of mossy fibers in the rat hippocampus. J. Neurosci. 18, 3386-3403.

Ahmad-Annuar, A., Ciani, L., Simeonidis, I., Herreros, J., Fredj, N. B., Rosso, S. B., Hall, A., Brickley, S., and Salinas, P. C. (2006). Signaling across the synapse: a role for Wnt and Dishevelled in presynaptic assembly and neurotransmitter release. J. Cell Biol. 174, 127-139.

Alvarez, V. A., and Sabatini, B. L. (2007). Anatomical and physiological plasticity of dendritic spines. Ann. Rev. Neurosci. 30, 79-97.

Ataman, B., Ashley, J., Gorczyca, M., Ramachandran, P., Fouquet, W., Sigrist, S. J., and Budnik, V. (2008). Rapid activity-dependent modifications in synaptic structure and function require bidirectional Wnt signaling. Neuron 57, 705-718.

Bakker, A., Kirwan, C. B., Miller, M., and Stark, C. E. (2008). Pattern separation in the human hippocampal CA3 and dentate gyrus. Science 319, 1640-1642.

Bamji, S. X., Rico, B., Kimes, N., and Reichardt, L. F. (2006). BDNF mobilizes synaptic vesicles and enhances synapse formation by disrupting cadherin-beta-catenin interactions. J.Cell Biol. 174, 289-299.

Barbosa, A. C., Kim, M. S., Ertunc, M., Adachi, M., Nelson, E. D., McAnally, J., Richardson, J. A., Kavalali, E. T., Monteggia, L. M., Bassel-Duby, R., and Olson, E. N. (2008). MEF2C, a transcritpion factor that facilitates learning and memory by negative regulation of synapse numbers and function. Proc. Natl. Acad. Sci. U.S.A. 105, 9391-9396.

Castillo, P. E., Janz, R., Sudhof, T. C., Tzounopoulos, T., Malenka, R. C., and Nicoll, R. A. (1997). Rab3A is essential for mossy fibre long-term potentiation in the hippocampus. Nature 388, 590-593.

Cerpa, W., Godoy, J. A., Alfaro, I., Ferias, G. G., Metcalfe, M. J., Fuentealba, R., Bonansco, C., and Inestrosa, N. C. (2008). Wnt-7a modulates the synaptic vesicle cycle and synaptic transmission in hippocampal neurons. J. Biol. Chem. 283, 5918-5927.

Chen, J., Park, C. S., and Tang, S. J. (2006). Activity-dependent synaptic Wnt release regulates hippocampal long term potentiation. J. Biol. Chem. 281, 11910-11916.

Chiu, S. L., Chen, C. M., and Cline, H. T. (2008). Insulin receptor signaling regulates synapse number, dendritic plasticity, and circuit function in vivo. Neuron 58, 708-719.

Chklovskii, D. B., Mel, B. W., and Svoboda, K. (2004). Cortical rewiring and information storage. Nature 431, 782-788.

Christopherson, K. S., Ullian, E. M., Stokes, C. C., Mullowney, C. E., Hell, J. W., Agah, A., Lawler, J., Mosher, D. F., Bornstein, P., and Barres, B. A. (2005). Thrombospondins are astrocyte-secreted proteins that promote CNS synaptogenesis. Cell 120, 421-433.

Claiborne, B. J., Amaral, D. G., and Cowan, W. M. (1986). A light and electron microscopic analysis of the mossy fibers of the rat dentate gyrus. J. Comp. Neurol. 246, 435-458.

Danzer, S. C., and McNamara, J. O. (2004). Localization of brain-derived neurotrophic factor to distinct terminals of mossy fiber axons implies regulation of both excitation and feedforward inhibition of CA3 pyramidal cells. J Neurosci. 15, 11346-11355.

Danzer, S. C., Kotloski, R. J., Walter, C., Hughes, M., and McNamara, J. O. (2008). Altered morphology of hippocampal dentate granule cell presynaptic and postsynaptic terminals following conditional deletion of TrkB. Hippocampus 18, 668-578.

Davis, G. W. (2006). Homeostatic control of neural activity: from phenomenology to molecular design. Ann. Rev. Neurosci. 29, 307-323.

De Paola, V., Arber, S., and Caroni P. (2003). AMPA receptors regulate dynamic equilibrium of presynaptic terminals in mature hippocampal networks. Nat. Neurosci. 6, 491-500.

Diano, S., Farr, S. A., Benoit, S. C., McNay, E. C., da Silva, I., Horvath, B., Gaskin, F. S., Nonaka, N., Jaeger, L. B., Banks, W. A., Morley, J. E., Pinto, S., Sherwin, R. S., Xu, L., Yamada, K. A., Sleeman, M. W., Tschop, M. H., and Horvath, T. L. (2006). Ghrelin controls hippocampal spine synapse density and memory performance. Nat. Neurosci. 9, 381-388.

Engel, D., and Jonas, P. (2005) Presynaptic action potential amplification by voltage-gated Na⁺ channels in hippocampal mossy fiber boutons. Neuron 45, 405-417.

Fuchs, E., Flugge, G., and Czeh, B. (2006). Remodeling of neuronal networks by stress. Front. Biosci. 11, 2746-2758.

Galimberti, I., Gogolla, N., Alberi, S., Santos, A. F., Muller, D., and Caroni, P. (2006). Long-term rearrangements of hippocampal mossy fiber connectivity in the adult regulated by experience. Neuron 50, 749-763.

Gogolla, N., Galimberti, I., and Caroni, P. (2007). Structural plasticity of axon terminals in the adult. Curr. Opin. Neurobiol. 17, 516-524.

Gonzales, R. B., DeLeon Galvan, C. J., Rangel, Y. M., and Claiborne, B. J. (2001). Distribution of thorny excrescences on CA3 pyramidal neurons in the rat hippocampus. J. Comp. Neurol. 430, 357-368.

Hall, A. C, Lucas, F. R., and Salinas, P. C. (2000). Axonal remodeling and synaptic differentiation in the cerebellum is regulated by WNT-7a signaling. Cell 100, 525-535.

Henze, D. A., Urban, N. N., and Barrionuevo, G. (2000). The multifarious hippocampal mossy fiber pathway: a review. Neuroscience 98, 407-427.

Henze, D. A., Wittner, L., Buzsaki, G. (2002). Single granule cells reliably discharge targets in the hippocampal CA3 network in vivo. Nat. Neurosci. 5, 790-795.

Huang, Y. Z., Pan, E., Xiong, Z. Q., and McNamara, J. O. (2008). Zinc-mediated transactivation of TrkB potentiates the hippocampal mossy fiber-CA3 pyramid synapse. Neuron 57, 546-558.

Ji, Y., Pang, P. T., Feng, L., and Lu, B. (2005). Cyclic AMP controls BDNF-induced TrkB phosphorylation and dendritic spine formation in mature hippocampal neurons. Nat.Neurosci. 8, 164-172.

Kavalali, E. T., Klingauf, J., and Tsien, R. W. (1999). Activity-dependent regulation of synaptic clustering in a hippocampal culture system. Proc. Natl. Acad. Sci U.S.A. 96, 12893-12900.

Kim, B. G., Dai, H. N., McAtee, M., and Bregman, B. S. (2008). Modulation of dendritic spine remodeling in the motor cortex following spinal cord injury: effects of environmental enrichment and combinatorial treatment with transplants and neurotrophin-3. J. Comp. Neurol. 508, 473-486.

Kobayashi, K., and Poo, M. M. (2004). Spike train timing-dependent associative modification of hippocampal CA3 recurrent synapses by mossy fibers. Neuron 41, 445-454.

Kwon, H. B., and Castillo, P. E. (2008). Long-term potentiation selectively expressed by NMDA receptors at hippocampal mossy fiber synapses. Neuron 57, 108-120.

Lawrence, J. J., and McBain, C. J. (2003). Interneuron diversity series: containing the detonation—feedforward inhibition in the CA3 hippocampus. Trends Neurosci. 26, 631-540.

Leranth, C., Petnehazy, O., and MacLusky, N. J. (2003). Gonadal hormones affect spine synaptic density in the CA1 hippocampal subfield of male rats. J. Neurosci. 23, 1588-1592.

Leutgeb, J. K., Leutgeb, S., Moser, M. B., and Moser, E. I. (2007). Pattern separation in the dentate gyrus and CA3 of the hippocampus. Science 315, 961-966.

Li, W., Howard, J. D., Parrish, T. B., and Gottfried, J. A. (2008). Aversive learning enhances perceptual and cortical discrimination of indiscriminable odor cues. Science 319, 1842-1845.

Lie, D. C., Colamarino, S. A., Song, H. J., Desire, L., Mira, H., Consiglio, A., Lein, E. S., Jessberger, S., Lansford, H., Deane, A. R., and Gage, F. H. (2005). Wnt signalling regulates adult hippocampal neurogenesis. Nature 437, 1370-1375.

Magariños, A. M., McEwen, B. S., Saboureau, M., and Pevet, P. (2006). Rapid and reversible changes in intrahippocampal connectivity during the course of hibernation in European hamsters. Proc. Natl. Acad. Sci. U.S.A. 103, 18775-18780.

McBain, C. J. (2008). Differential mechanisms of transmission and plasticity at mossy fiber synapses. Progr. Brain Res. 169, 225-240.

McEwen, B. S. (1999). Stress and hippocampal plasticity. Annu.Rev.Neurosci. 22, 105-122.

McHugh, T. J., Jones, M. W., Quinn, J. J., Balthasar, N., Coppari, R., Elmquist, J. K., Lowell, B. B., Fanselow, M. S., Wilson, M. A., and Tonegawa, S. (2007). Dentate gyrus NMDA receptors mediate rapid pattern separation in the hippocampal network. Science 317, 94-99.

Mikels, A. J., and Nusse, R. (2006). Wnts as ligands: processing, secretion and reception. Oncogene 25, 7461-7468.

Mori, M., Abegg, M. H., Gahwiler, B. H., and Gerber, U. (2004). A frequency-dependent switch from inhibition to excitation in a hippocampal unitary circuit. Nature 431, 453-456.

Moser, M. B., Trommald, M., Egeland, T., and Andersen, P. (1997). Spatial training in a complex environment and isolation alter the spine distribution differently in rat CA1 pyramidal cells. J. Comp. Neurol. 380, 373-381.

Nicoll, R. A, and Schmitz, D. (2005). Synaptic plasticity at hippocampal mossy fibre synapses. Nat. Rev. Neurosci. 6, 863-876.

Ninkovic, J., Mori, T., and Götz, M. (2007). Distinct modes of neuron addition in adult mouse neurogenesis. J. Neurosci. 27, 10906-10911.

Packard, M., Koo, E. S., Gorczyca, M., Sharpe, J., Cumberledge, S., and Budnik, V. (2002). The Drosophila Wnt, wingless, provides an essential signal for pre- and postsynaptic differentiation. Cell 111, 319-330.

Pleskacheva, M. G., Wolfer, D. P., Kupriyanova, I. F., Nikolenko, D. L., Scheffrahn, H., Dell'Omo, G., and Lipp, H. P. (2000). Hippocampal mossy fibers and swimming navigation learning in two vole species occupying different habitats. Hippocampus 10, 17-30.

Polley, D. B., Kvasnák, E., and Frostig, R. D. (2004). Naturalistic experience transforms sensory maps in the adult cortex of caged animals. Nature 429, 67-71.

Ramirez-Amaya, V., Balderas, I., Sandoval, J., Escobar, M. L., and Bermudez-Rattoni, F. (2001). Spatial long-term memory is related to mossy fiber synaptogenesis. J. Neurosci. 21, 7340-7348.

Rebola, N., Lujan, R., Cunha, R. A., and Mulle, C. (2008). Adenosine A_{2A} receptors are essential for long-term potentiation of NMDA-EPSCs at hippocampal mossy fiber synapses. Neuron 57, 121-134.

Rich, M. M., and Wenner, P. (2007). Sensing and expressing homeostatic synaptic plasticity. Trends Neurosci. 30, 119-125.

Rollenhagen, A. Satzler, K., Rodriguez, E. P., Jonas, P., Frotscher, M., and Lübke, J. H. (2007). Structural determinants of transmission at large mossy fiber synapses. J. Neurosci. 27, 10434-10444.

Rosso, S. B., Sussman, D., Wynshaw-Boris, A., and Salinas, P. C. (2005). Wnt signaling through Dishevelled, Rac and JNK regulates dendritic development. Nat. Neuroci. 8, 34-42.

Salinas, P. C. (2005). Retrograde signalling at the synapse: a role for Wnt proteins. Biochem. Soc. Trans. 33, 1295-1298.

Salinas, P. C., and Zou, Y. (2008). Wnt signaling in neural circuit assembly. Ann. Rev .Neurosci. 31, 339-358.

Sandi, C., Davies, H. A., Cordero, M. I., Rodriguez, J. J., Popov, V. I., and Stewart, M. G. (2003). Rapid reversal of stress induced loss of synapses in CA3 of rat hippocampus following water maze training. Eur. J. Neurosci. 17, 2447-2456.

Sato, K., Akaishi, T., Matsuki, N., Ohno, Y., Nakazawa, K. (2007). beta-Estradiol induces synaptogenesis in the hippocampus by enhancing brain-derived neurotrophic factor release from dentate gyrus granule cells. Brain Res. 1150, 108-120.

Schopke, R., Wolfer, D. P., Lipp, H. P., and Leisinger-Trigona, M. C. (1991). Swimming navigation and structural variations of the infrapyramidal mossy fibers in the hippocampus of the mouse. Hippocampus 1, 315-328.

Stewart, M. G., Davies, H. A., Sandi, C., Kraev, I. V., Rogachevsky, V. V., Peddie, C. J., Rodriguez, J. J., Cordero, M. I., Donohue, H. S., Gabbott, P. L., and Popov, V. I. (2005). Stress suppresses and learning induces plasticity in CA3 of rat hippocampus: a three-dimensional ultrastructural study of thorny excrescences and their postsynaptic densities. Neuroscience 131, 43-54.

Stoppini, L., Buchs, P. A., and Muller, D. (1991). A simple method for organotypic cultures of nervous tissue. J. Neurosci. Methods 37, 173-182.

Dieck T., S., Sanmarti-Vila, L., Langnaese, K., Richter, K., Kindler, S., Soyke, A., Wex, H., Smalla, K. H., Kampf, U., Franzer, J. T., Stumm, M., Garner, C. C., and Gundelfinger, E. D. (1998). Bassoon, a novel zinc-finger CAG/glutamine-repeat protein selectively localized at the active zone of presynaptic nerve terminals. J. Cell Biol. 142, 499-509.

van Praag, H., Kempermann, G., and Gage, F. H. (2000). Neural consequences of environmental enrichment. Nat. Rev. Neurosci. 1, 191-198.

Wayman, G. A., Impey, S., Marks, D., Saneyoshi, T., Grant, W. F., Derkach, V., and Soderling, T. R. (2006). Activity-dependent dendritic arborization mediated by CaM-kinase I activation and enhanced CREB-dependent transcription of Wnt-2. Neuron 50, 897-909.

Yankova, M., Hart, S. A., and Woolley, C. S. (2001). Estrogen increases synaptic connectivity between single presynaptic inputs and multiple postsynaptic CA1 pyramidal cells: a serial electron-microscopic study. Proc. Natl. Acad. Sci. U.S.A. 98, 3525-3530.

1. A method of preventing and/or treating a disease characterized by a loss or imbalance of neuronal connections in a subject by administering to said subject a therapeutically effective amount of a modulator of Wnt-signaling, wherein
(i) said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, and wherein
(ii) said subject is in puberty or post-puberty, wherein puberty or post-puberty means that the neuronal circuits of said subject have already undergone developmental synapse elimination and critical periods which take place prior to puberty.

2. A method of modulating neuronal activity and/or neuronal plasticity, optimizing repair after brain injury, modulating learning, and/or modulating cognitive capabilities in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty.

3. A method of preventing loss of neuronal connections and/or increasing neuronal connections in a subject by administering to said subject an effective amount of a modulator of Wnt-signaling, wherein said subject is in puberty or post-puberty, wherein puberty or post-puberty means that the neuronal circuits of said subject have already undergone developmental synapse elimination and critical periods which take place prior to puberty.

4. The method of claim 1 wherein Wnt-signaling is modulated by an agonist.

5. The method of claim 4 wherein the agonist is an antibody.

6. The method of claim 4 wherein the agonist decreases or silences the expression of an antagonistic component of Wnt-signaling.

7. The method of claim 6 wherein the agonist is a siRNA.

8. The method of claim 1 wherein the subject is a mammal.

9. The method of claim 1 wherein the modulator acts in the hippocampus of said subject.

10. The method of claim 1 wherein the expression and/or biological activity of Wnt7a and/or Wnt7b is modulated or inhibited by the modulator.

11. (canceled)

12. (canceled)

13. The method of claim 5, wherein said antibody inhibits the binding of said Wnt-signaling component to its ligand.

14. A drug delivery device for implantation into the nervous system of a subject who is in puberty or post-puberty, wherein puberty or post-puberty means that the neuronal circuits of said subject have already undergone developmental synapse elimination and critical periods which take place prior to puberty, the drug delivery device comprising: a biocompatible polymer; and a modulator, of Wnt-signaling, wherein said modulator of Wnt-signaling is released from the polymer into the nervous system of the subject in an amount effective to treat or prevent a disease characterized by a loss or imbalance of neuronal connections, wherein said disease characterized by a loss or imbalance of neuronal connections is selected from a group of diseases consisting of age-related loss of neuronal performance and/or connections, psychiatric disorders, neurodegenerative diseases characterized by loss of neuronal connections, learning deficiency, and senile dementia, to modulate neuronal activity and/or neuronal plasticity, to optimize repair after brain injury, to modulate learning, and/or to modulate cognitive capabilities, in said subject and/or, to prevent loss of neuronal connections and/or to increase neuronal connections in said subject.

15. The method of claim 4, wherein the agonist is a small molecule.

16. The method of claim 8, wherein the mammal is a human.

17. The method of claim 2, wherein the modulator is a siRNA.

18. The method of claim 3, wherein the modulator is a siRNA.

19. The method of claim 2, wherein the modulator is an antibody.

20. The method of claim 3, wherein the modulator is an antibody.
